(19) United States
(12) Patent Application Publication
(10) Pub. No.: US 2023/0136014 A1
(43) Pub. Date: May 4, 2023

(54) PROCESS AND APPARATUS FOR THREE-STAGE BIOLOGICAL PARTICULATE ELIMINATOR

(71) Applicant: Pathogen Protection Applications, LLC, Edmond, OK (US)

(72) Inventors: Donald R. Pyron, Camden, AR (US); H. Andrew Pyron, Yankton, SD (US)

(21) Appl. No.: 17/423,313

(22) PCT Filed: Jul. 2, 2021
(86) PCT No.: PCT/US2021/040304

§ 371 Date: Jul. 15, 2021

Related U.S. Application Data
(60) Provisional application No. 63/047,488, filed on Jul. 2, 2020.

(51) Int. Cl. F24F 8/20; F24F 8/22; A61L 9/03; A61L 9/20

(52) U.S. Cl. CPC F24F 8/20; F24F 8/22; A61L 9/032; A61L 9/20; F24F 2221/34; A61L 2209/16; A61L 2209/12; A61L 2209/14

(57) ABSTRACT

An air treatment system includes an air duct separate from any existing HVAC system. A burner is communicated and is configured to heat the air received in the air duct to a temperature sufficient to reduce the pathogen load of the air passing therethrough. A cooling apparatus will create a cool air stream having a temperature of not more than 10° F. higher than the ambient temperature of the indoor space from which the air was received.

### CROSS-REFERENCE TO RELATED APPLICATIONS

This application claims the benefit of PCT Patent Application No. PCT/US2021/040304 filed Jul. 2, 2021, and U.S. Provisional Application No. 63/047,488 filed Jul. 2, 2020, which are hereby incorporated by reference.

It is well known that pathogens, allergens and other airborne contaminants including pathogenic microbes, pollutants, viruses and other microorganisms cause a number of health hazards. In particular, in indoor spaces each time an occupant of that indoor space exhales or sneezes, microorganisms are carried into the indoor air that is ultimately breathed in by others. With the recent outbreak of COVID-19, which is caused by the SARS-CoV-2 virus, the importance of reducing, and if possible removing pathogens from indoor environments in which humans live, work and otherwise occupy has received much recent attention. Known efforts to reduce pathogens and/or microbes in an indoor environment typically are associated with the use of components connected to and used within existing HVAC systems that supply air to an indoor space.

### DESCRIPTION OF THE DRAWINGS

FIG. 1 schematically shows an arrangement of an air treatment system for an indoor space.

FIG. 2 shows a cross section of a burner configuration in the embodiment of FIG. 1.

FIG. 3 is a perspective view of an embodiment of an updraft hood utilized with the system of FIG. 1.

FIG. 4 schematically shows a portion of the system of FIG. 1 with a heat exchanger included.

FIG. 5 is a schematic of an additional embodiment system for treating air.

FIG. 6 schematically shows an example of an indoor space with which the systems and methods disclosed and claimed may be used.

FIG. 7 is a close-up view of an embodiment of updraft hoods positioned at work spaces in an interior of a building.

FIG. 8 is an additional building configuration.

FIG. 9 is a view identical to that in FIG. 8 but includes embodiments of updraft hoods for human occupiable spaces.

FIG. 10 shows an embodiment of a heating arrangement for the system.

FIG. 11 is a partial interior view of one of the silos that may be utilized with the system of FIG. 10.

FIG. 12 shows the use of an induction coil proximate a burner cylinder housing.

FIG. 13 is a partial cross section of an individual burner cylinder used with the embodiment of FIG. 10.

FIG. 14 shows an additional indoor space.

FIG. 15 schematically shows a test arrangement of the system as described herein.

FIG. 16 are results of tests conducted with the arrangement of FIG. 15.

### DESCRIPTION OF AN EMBODIMENT

Embodiments herein include systems and methods for reducing the pathogen load in an indoor space. Embodiments also include systems and methods for preventing air expelled by humans from entering an indoor space. The systems and methods described herein include systems for eliminating, or at least reducing pathogens including harmful microbes, viruses and other life-threatening pathogens in an indoor space. Pathogens, as used herein, is intended to include harmful microbes and viruses including but not limited to SARS CoV-2. The systems and methods described herein operate independent of existing HVAC systems used to condition the air in an indoor space, and do not connect to or use the ductwork or other components of HVAC systems.

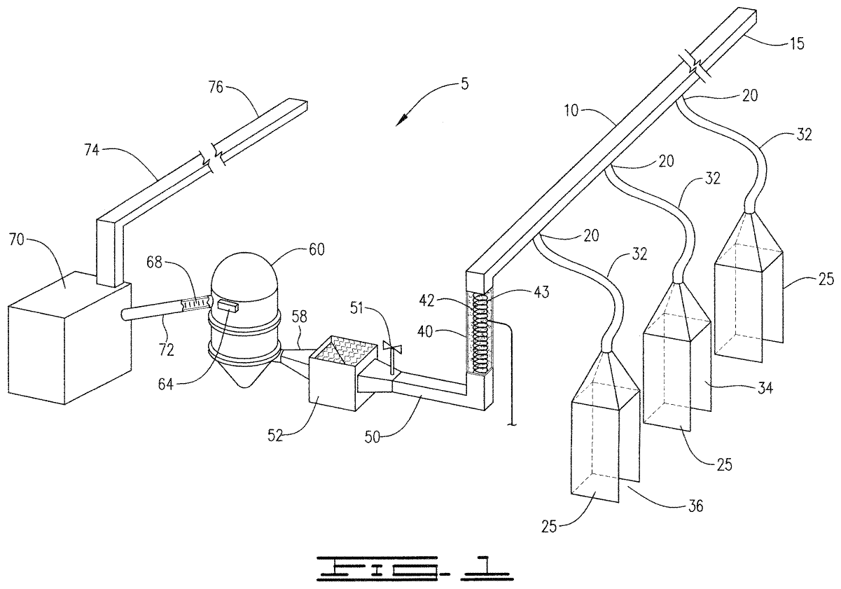

Referring now to the figures, FIG. 1 schematically shows an air treatment system 5 configured to prevent pathogens expelled by humans from entering indoor spaces, and to eliminate, or at least reduce pathogens from the indoor space 6. The air in the indoor space 6, including air expelled by humans may at times be referred to as contaminated air. The system 5 was developed to target SARS-CoV-2. However, the usefulness of the system is not limited to SARS-CoV-2 and the system may be utilized to remove not only SARS-CoV-2 but other pathogens. In the embodiment of FIG. 1, system 5 includes an air duct 10 with a closed end 15 and a plurality of air inlets 20.

System 5 further includes a plurality of updraft hoods 25 which may be referred to as updraft cubicles. Air conduits 32 connect updraft hoods 25 with the air duct 10 through inlets 20 defined therein. In the embodiment described updraft hoods 25 may comprise updraft cubicles with an outer wall 34 and an open bottom 36. A side opening 38 is defined in the outer wall which will allow the entry and exit of a human operator positioned at a work station or other location occupiable by humans. The updraft cubicle in the described embodiment is generally rectangularly shaped in cross section, but is not limited to such a shape. If desired, updraft hoods 25 may have a curtain or wall that covers the opening 38 such that the only opening for the updraft hoods 25 once occupied is the open bottom 36.

A burn zone 40 is communicated with air duct 10. Burn zone 40 may comprise a burner 42 with outer housing 48 that defines an interior 43 therein. Burner 42 is heatable to a high temperature. In one embodiment burner 42 may comprise a media 44 in an outer housing 48. The media will be of a type that can be heated to and maintain high temperatures of as much as 500° F. In some embodiments the media can be heated to as much as 600° F., 700° F., 800° F., 900° F. and up to 1000° F. The media may comprise for example a ceramic material or metal beads. The media will in all cases be porous, or will otherwise define a travel path through the burner 42 to allow the flow of air therethrough.

A heating element 46 such as for example an electric coil may also be disposed in the outer housing 48 of burner 42. Other known means of heating may be utilized, such as an induction coil placed proximate a metal rod or other metal object placed in the interior 43. Outer housing 48 can be comprised of a material that will withstand high temperatures without emitting excessive heat to the surrounding area. The outer housing may also have an insulating material therearound to insulate burner 42. The heated air stream leaving burner 42 will have reduced pathogens as compared to the contaminated air entering the burner 42, and will be cooled as described below.

A connecting duct 50 communicates burn zone 40 with a scrubber 52. Scrubber 52 serves a dual purpose in that air passing therethrough will go through a spray of vapor in one embodiment, or in another embodiment will pass through a fill media over which water circulates. In this manner the air can be cooled and filtered at the same time. The water may in some cases be chemically treated such that it may comprise for example chlorinated water at pH levels that are equal or similar to those recommended for public swimming pools. Scrubber 52 is therefore also referred to as a cooling apparatus. A pump (not shown) may circulate water across a fill media in scrubber 52 to allow the air to be cooled and scrubbed. It is not necessary to provide any chemical treatment to the water such that the water may simply be used as a cooling agent. A fresh air damper 51 may be included in connecting duct 50. Fresh air from the indoor space may be drawn into the connecting duct 50 through fresh air damper 51 to aid in cooling the heated air stream exiting burner 42.

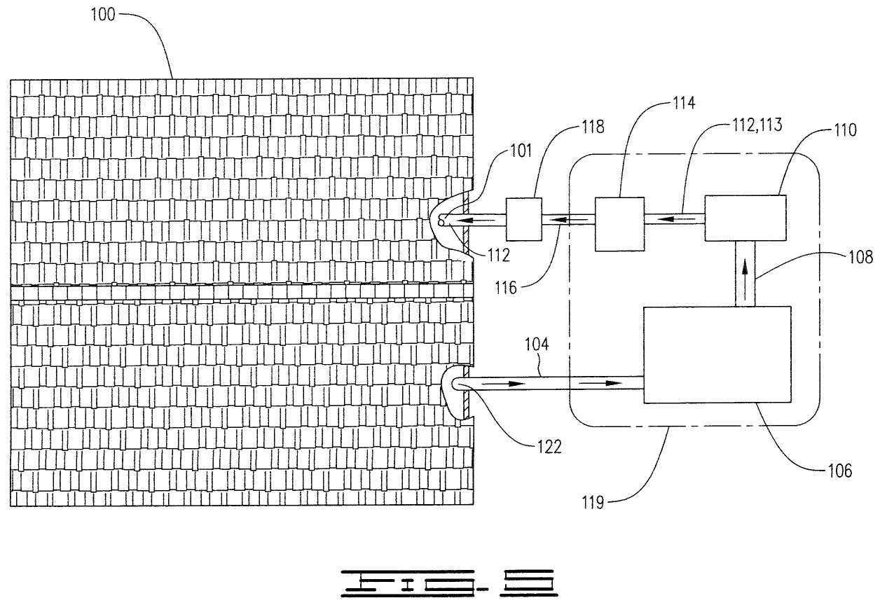

Additional cooling may be supplied by a heat exchanger 56 as schematically shown in FIG. 4, Heat exchanger 56 may be of a type known in the art. For example, heat exchanger 56 may comprise a plurality of tubes in which a cooling agent passes. In one embodiment the cooling agent may be cool water circulated from for example an exterior cooling tower, Cool water will pass through tubes over which heated air leaving burner 42 will flow to be cooled. The water will be directed out to an exterior tower through a flow conduit 57 where it will be cooled and recirculated back into the heat exchanger 56 through a conduit 59. If desired, the water used in scrubber 52 may be circulated through an exterior cooling tower using flow conduits 53 and 55. Water will flow in the direction of the arrows. A connecting conduit 58 communicates scrubber 52 with a UV chamber 60.

A cooled air stream leaves scrubber 52 and enters the UV chamber at a tangent 62 so that a centrifugal motion is created. Air will move in a centrifugal manner around the inner wall of the UV chamber 60. Ultraviolet lights 64 may be inserted and extend into an interior 61 of UV chamber 60. UV chamber 60 may also include a plurality of UV lights arranged in a spiral fashion along the interior 61. In the schematic of FIG. 4 an upper portion of UV chamber 60 is not shown so that interior 61 may be seen. Air passing through the UV chamber 60 is thus directly exposed to UV radiation prior to exiting into the indoor space. The UV lamps may comprise UVC lamps with ozone. A pleated filter which may be for example a dust collection, or particulate filter 68 may be positioned at an exit of the UV chamber 60 to collect any particulates that might exist in the cooled air stream passing through the UV chamber 60.

A connecting conduit 72 communicates UV chamber 60 with a blower, or pump 70 of a type known in the art. Blower 70 will operate to create a negative pressure, or soft vacuum at air inlets 20. As a result, air will be pulled into air duct 10 through updraft hoods 25. Updraft hoods 25 are positioned at human occupiable locations. Human occupiable locations as used herein means those places in a room or building that in a typical situation are occupied by humans, such as a work station, a desk, seating at a restaurant or in an office area. By strategically locating updraft hoods 25 at such locations, the system 5 can prevent pathogen laden air expelled by a human in those locations from ever entering the indoor space outside the updraft hood. At least a portion of the air expelled by the human will be contained within the updraft hood and passed into the system 5 for treatment as described, and in some cases all of the air expelled by the human will be contained within the updraft hood and passed into the system 5.

A human worker in the updraft hood 25 will expel air through normal exhalation, coughing, sneezing, talking and other bodily functions that require air to be expelled through the nose or the mouth. The vacuum created by the pump 70 should be at a level sufficient to pull the majority of the air, and in some cases all of the air expelled by the human positioned in the updraft hood 25. Any pathogens in the expelled air pulled into the air duct 10 will be treated and conditioned as described. In this way, pathogens expelled by humans are prevented from ever entering indoor space 6 and contaminating the air therein. Air is pulled into air duct 10 and communicated through the burn zone 40. In one embodiment temperature in the burn zone exceeds 500° F. degrees. In additional embodiments the temperature in the burn zone is between 500 and 1,000° F. In other embodiments, the temperature may be between 500° F. and 900° F., 500° F. and 800° F., 500° F. and 700° F., and 500° F. and 600° F. and any ranges therebetween. In any event, burner 42 is configured to heat the burn zone to an internal temperature of as much as 1,000° F. The heated air stream leaving the burner 42 may likewise reach the foregoing temperatures. The treated air leaving burner 42 will have a reduced pathogen load as compared to the contaminated air. The cooled, treated air stream is delivered into the indoor space through an air exit 74 at, or near the ambient temperature of the indoor space.

As is understood from the drawings a method of removing pathogens may comprise positioning a plurality of air inlets at a plurality of locations in the indoor space. The method may further include pulling indoor air into the air inlets 20 and passing the indoor air through a burner 42 having an internal temperature sufficient to eliminate, or at least significantly reduce pathogens in the air passing through the burner. The method may comprise exposing the air to high temperatures for a short period of time at the high temperature. In one embodiment the method may comprise exposing the air in a burner to temperature ranges noted above for no more than three seconds to eliminate, or at least reduce the pathogens that existed in the air prior to the exposure to the temperatures in the burner. In a specific embodiment, the method includes exposing the air to a temperature range of between about 517° F. and 662° F. for no more than 2.61 seconds.

The method may comprise passing the indoor air through a burner having an internal temperature of at least 500° F. to create a heated air stream. The method may comprise heating the indoor air to higher temperatures, for example 600° F., 700° F., 800° F., 900° F. or 1000° F. to create a heated air stream. The heated air stream may then be cooled to create a cooled air stream having a temperature about the same as the air in the indoor space. In one embodiment the temperature of the air will be not more than 10° F. different than the temperature of the air in the indoor space. The cooled air stream is then exhausted back into the indoor space. The method may further comprise exposing a cooled air stream to UV radiation prior to the exhausting step.

The cooling step may comprise for example injecting a vapor stream into the heated air stream after it leaves the burner, and/or passing the air through a heat exchanger and/or through a scrubber with fill media over which cool water flows. The method may also include preventing pathogens expelled from a human from entering indoor air space 6. The method includes placing an updraft hood 25 around at least the head of a human in the indoor space, and pulling the air emitted by the human from the updraft hood 25 through an air inlet into an air duct that communicates the air into the burner 42, The air is then treated and conditioned as described and is exhausted as treated air into the indoor air space. The treated air will be clean air, and will be free of pathogens or will at least have a reduced level of pathogens than existed in the contaminated air.

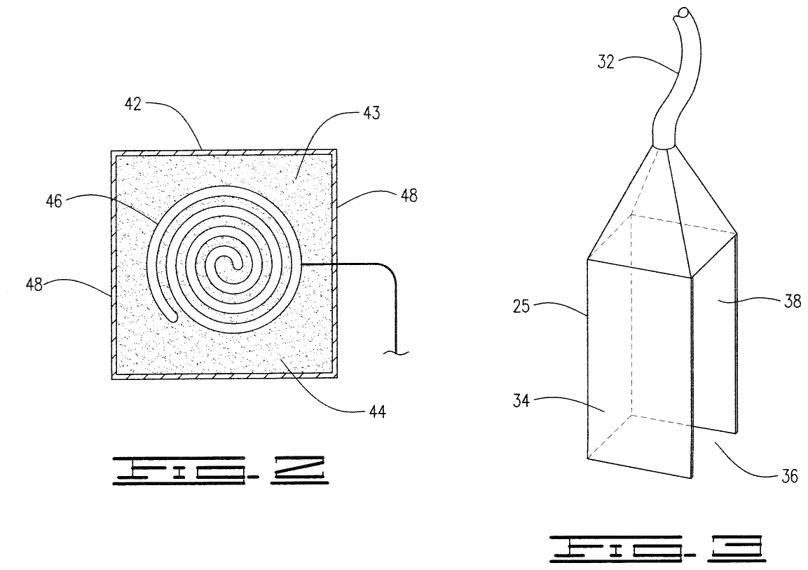

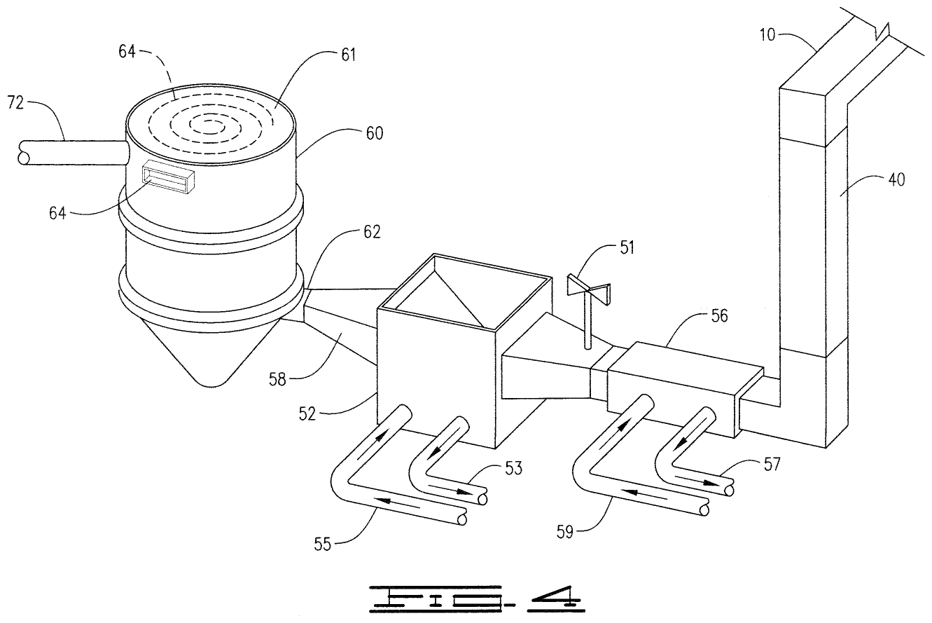

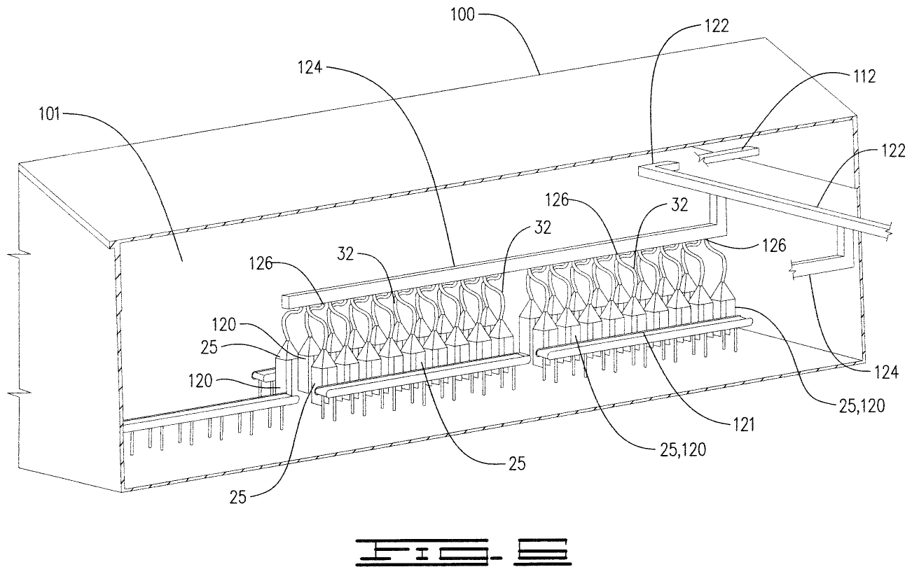

In the embodiment described in FIGS. 1-3 the system 5 is shown as contained within the indoor space 6 in which the air is being treated. As noted above, the entire system 5 operates completely independent of existing HVAC systems, and is not connected thereto in any way. Other embodiments may be utilized on a larger scale and may include multiple air ducts and a greater plurality of updraft hoods than shown in FIG. 1. For example, FIG. 5 schematically shows a building which may be a large or small building having an enclosed indoor space 101. Air is directed from indoor space 101 in building 100 to an air treatment system 102 configured to prevent contaminated air expelled from humans from entering the indoor space, and to eliminate, or at least significantly reduce pathogens from the air within indoor space 101. Air treatment system 102 is shown in FIG. 5 positioned outside building 100.

Air may be communicated from indoor air space 101 through an air supply duct 104. Air will pass through a burner 106 which will reach temperatures sufficient such that pathogens in the air passing therethrough, such as in a non-limiting example SARS-CoV-2, will be eliminated or at least significantly reduced. A connecting conduit 108 connects burner 106 with a cooling apparatus 110. Cooling apparatus 110 may be for example a cooling tower with media over which water is sprayed. One embodiment may have a closed circuit cooling tower which may include a coil through which the heated air stream from burner 106 flows. Water will pass over the coils in the cooling tower to cool the heated air stream so that a cooled air stream exits cooling apparatus 110. Other cooling apparatus may be utilized to cool the heated air to create a cooled, treated air steam that will exit cooling apparatus 110 through a return air conduit 112. A UV chamber 114 includes a plurality of UV lamps, which may be UVC lamps, positioned therein. UV chamber 114 is connected in return air conduit 112. Return air conduit 112 may be divided into a first portion 113 and a second portion 116 so that the UV chamber separates the return air conduit into two portions. The cooled, treated air stream will pass through first portion 113 in return air conduit 112 and into the second portion 116 through UV chamber 114 where the air passing therethrough is directly exposed to UV radiation. The cooled air stream thus passes from return air conduit 112 into the indoor space 101. The cooled air stream entering the indoor space will be at about the same temperature of the air in the indoor space. In one embodiment the cooled air stream is at a temperature of no greater than 10° F. above the ambient air temperature in the indoor air space and in another no greater than 5° F.

An air pump 118 may be used to create a vacuum to pull the contaminated air from updraft hoods in the indoor space 101 through the system 102. All, or part of the system components including burner 106, cooling apparatus 110, UV chamber 114 and air pump 118 may be housed in a system building 119, or may be separately housed or simply positioned exterior to building 100.

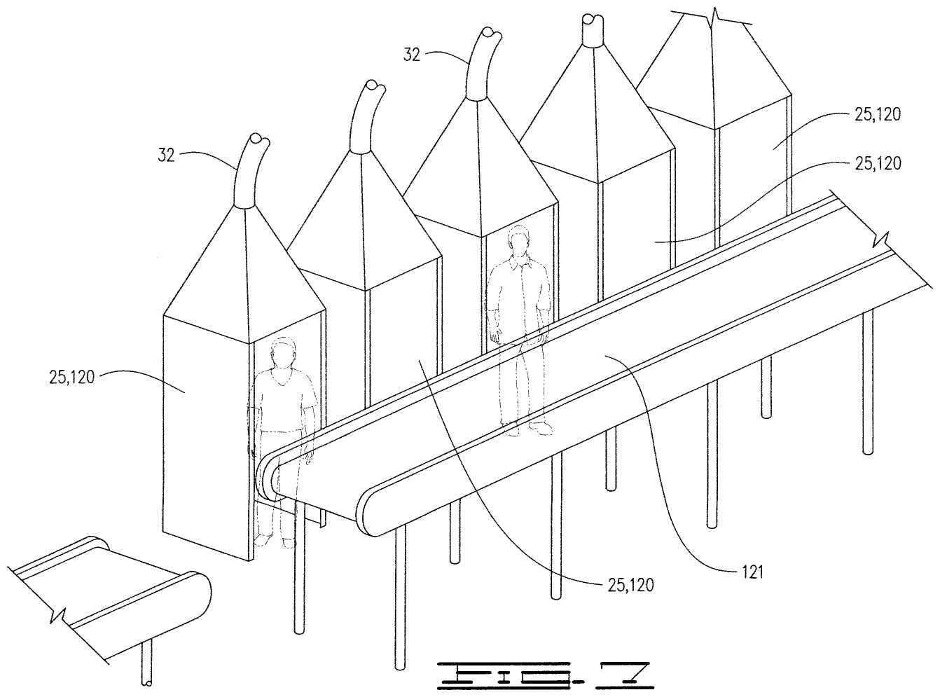

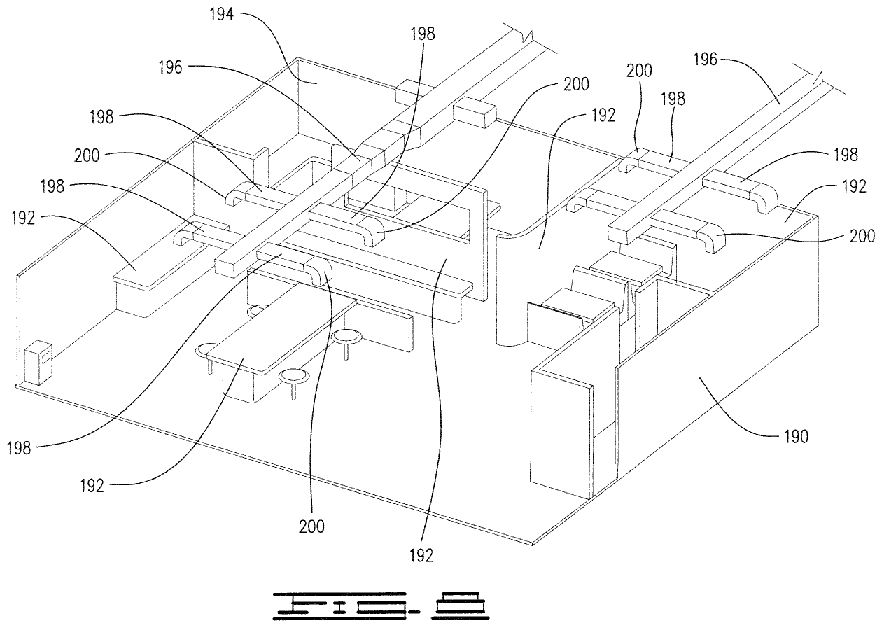

As schematically depicted in FIG. 6, building 100 may be for example an industrial building with a plurality of human occupiable locations 120, which may be work stations 120. Work stations 120 may be for example adjacent a conveyor belt 121 or other equipment. Air supply duct 104 has indoor air duct 122 connected thereto. Indoor air duct 122 may have a plurality of air duct branches 124 extending therefrom with a plurality of air inlets 126. A plurality of updraft hoods 25 are connected by air conduits 32 to the indoor air duct 122. Only one air duct branch 124 is shown with updraft hoods 25 connected thereto, but it is understood that a plurality of air duct branches 124 may be so configured. As shown in FIGS. 6 and 7 updraft hoods 25 may extend all the way to the ground surface such that a human operator positioned at a human occupiable space 120 is partially enclosed from the ground surface upward. The updraft hood 25 will in some cases be above the head of a human, and in others may only extend downward to partially enclose an upper portion of a human to below the mouth and nose. If desired, a completely closed updraft hood may be created by simply providing a wall or curtain on the open portion 38 of the outer wall.

The operation of system 102 is generally the same as that with respect to system 5 only on a larger scale. Air pump 118 will create negative pressure at each of the air inlets 126 through conduits 32 and will pull air upwardly from updraft hoods 25 into duct branches 124 and air duct 122. Thus, air expelled by sneezes, exhalations or otherwise by any human operators in the updraft hoods 25 will be pulled upwardly along with any air that is pulled through the opening 38 from indoor space 101. As a result, the system 102 is not only a system that eliminates, or reduces pathogens, but is a system and method that prevents pathogens expelled by humans from entering and contaminating the air in indoor space 101. Air expelled by humans in updraft hoods 25, along with air from indoor space 101 that passes into updraft hoods 25 through the open side thereof, will pass through air duct 122 into the supply air duct 104 and into system 102. Burner 106 is configured such that the internal temperature will reach at least 500° F. In other embodiments the burner 106 is configured to reach internal temperatures of at least 600° F., 700° F., 800° F., 900° F., and as much as 1,000° F. Air passing though burner 106 will be exposed to the temperature in the burner 106, and exposing air to the temperatures described for burner 106 will reduce, and in most cases eliminate pathogens from the air passing therethrough. Based on the below described testing conducted on the impact of increased temperatures on MS-2, which is a surrogate for SARS-CoV-2, it is believed that exposing contaminated air to -elevated temperatures for a short period of time, for example as little as five seconds, and further as little as three seconds, will kill pathogens, and more specifically will kill SARS-CoV-2. It is likewise believed that the higher the temperature, the less residence time will be needed. Thus, it is believed that exposing the contaminated air to temperatures of above 500° F., for example as much 600° F., 700° F., 800° F., 900° F., and/or 1,000° F., and temperature ranges therebetween, may kill pathogens in contaminated air, and will do so with little residence, or exposure time. In one embodiment, air passing through a burner is exposed to an entry temperature of about 517° F. at the point of entry for the air into the burner and an exit temperature is about 662° F. The residence time for air passing through the burner may be less than three seconds, and in one example about 2.61 seconds.

A heated air stream will leave burner 106 and will begin to cool in the connecting duct 108 from the burner 106 to the cooling apparatus 110. The heated air stream will be cooled further by the cooling apparatus 110. The heated air stream will be cooled by cooling apparatus 110 to a temperature of not greater than 10° F. and preferably not greater than 5° F. over the ambient air temperature in the indoor space.

In one embodiment the updraft hoods 25 that are not occupied can be deactivated. One manner of doing this would be simply to have a valve in the conduits 32 that can be automatically controlled from a controller. In this way less power will be required to generate the air flow necessary to pull the air from activated updraft hoods. In addition, an updraft hood 25 can be movable from a lowered position in which it at least partially covers a human, to a raised position in which the updraft hood 25 is positioned above a human in the occupiable space 120.

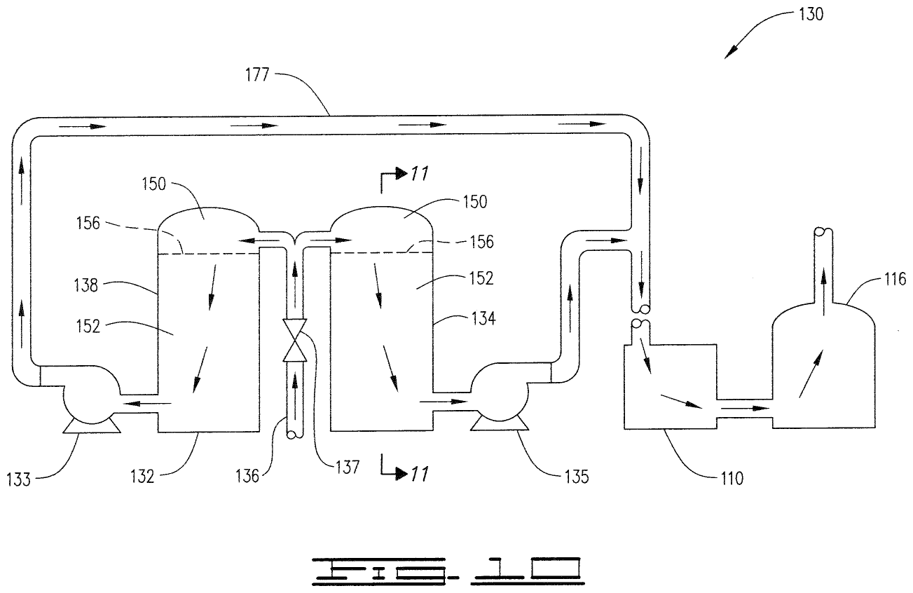

Burner 106 can be for example a plurality of burners 42 as described earlier connected in series. Burner 106 may have other configurations capable of reaching the internal temperatures described herein. An example of a burner embodiment may be as described with respect to FIGS. 10-13. As shown therein, burner assembly 130 comprises a pair of burners 132 and 134. Burners 132 and 134 are generally identical in construction. Burners 132 and 134 have pumps 133 and 135 respectively communicated therewith. Air is received from the inner air space through air supply duct 104. Air will flow into air supply duct 104 from indoor air duct 122 and into burner 106. A burner inlet conduit 136 receives air from air supply duct 104. Burner inlet conduit 136 may have a valve 137 therein that will allow the air received therein to be directed to either of burners 132 or 134. Each of burners 132 and 134 comprise an outer housing or silo 138 having an interior 140.

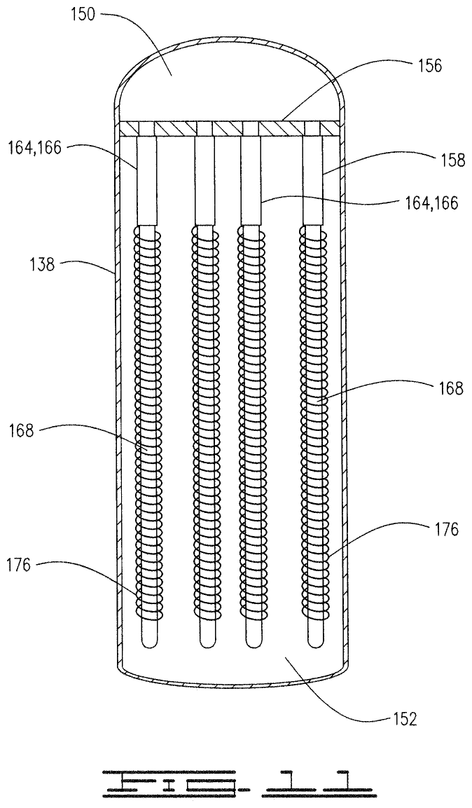

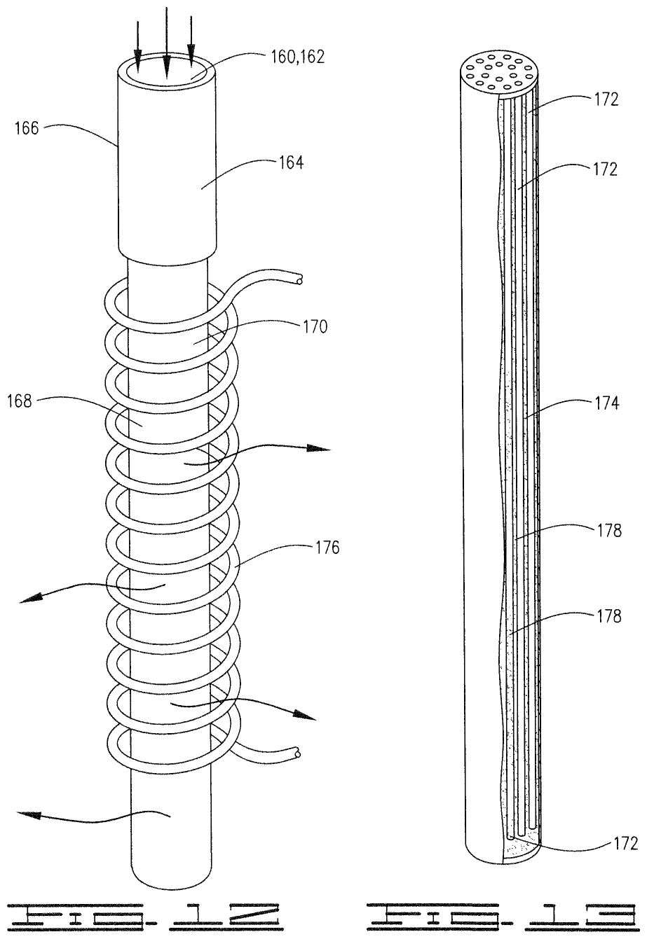

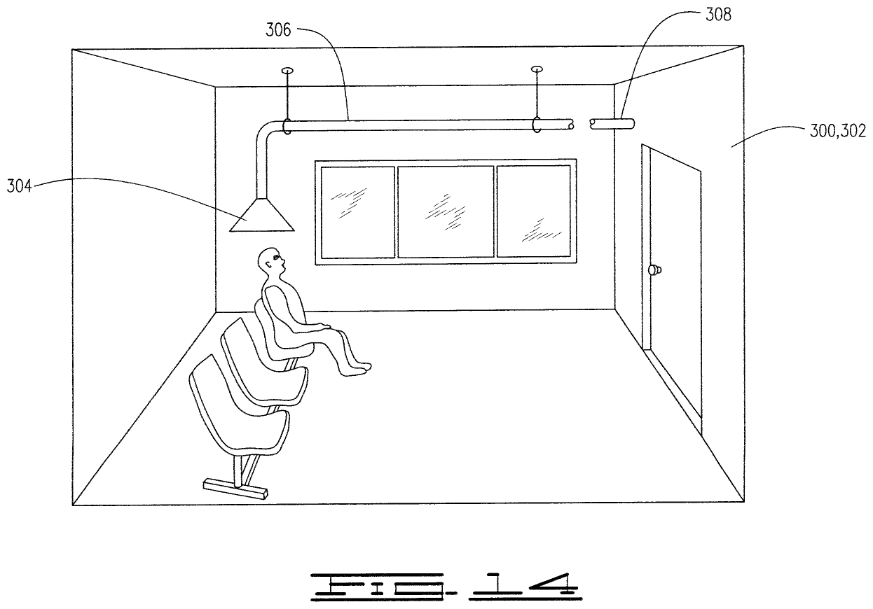

Interior 140 may be separated into a dirty air plenum 150 in an upper portion thereof and a clean air plenum 152 in a lower portion thereof. In one embodiment dirty air plenum 150 may comprise a heated plenum. Dirty air plenum 150 may be heated by a gas fired fire box or other known methods. FIG. 11 shows a view of the interior 140 of a silo 138. A tube sheet 156 divides the interior into the dirty air and clean air plenums 150 and 152, respectively. Tube sheet 156 will have a plurality of openings through which individual burner cylinders 158 may be suspended. Only four burner cylinders 158 are shown in the view of FIG. 11 but it is understood that more or less may be suspended from tube sheet 156 depending upon the amount of air flowing therethrough to be heated. Referring to burner 132, pump 133 will create negative pressure to pull air into silo 138 of burner 132 from air supply conduit 104. Air will pass from dirty air plenum 150 into the individual burner cylinders 158. As depicted in FIGS. 12 and 13, individual burner cylinders have an opening 160 at an upper end 162 thereof. A shield 164 may be disposed about an upper portion 166 of burner cylinders 158 to prevent air flowing therein from passing outward prior to the time the air passes through the burner portion 168 of burner cylinder 158.

Burner portion 168 has an outer housing 170 which may be a porous outer housing 170. In one embodiment the outer housing 170 may comprise a ceramic material. In the partial section view shown in FIG. 13, a plurality of metal rods 172 are shown disposed in an interior 174 of outer housing 170. A heating coil 176, which may be for example an electric coil or an induction coil is disposed about outer housing 170 and is proximate metal rods 172. In the embodiment shown the induction coil 176 is wrapped about outer housing 170 and is positioned close enough to metal rods 172 such that when a current is applied thereto induction coil 176 will generate significant heat which will heat metal rods 172 and consequently fill media 178.

Outer housing 170 is filled with fill material 178 through which metal rods 172 extend. The fill material 178 may comprise metal beads, such as stainless steel beads or may comprise a ceramic or other material that can withstand temperatures of the ranges discussed herein and allow air to pass therethrough. The internal temperature of each of burner cylinder 158 will reach a minimum of at least 500° F., and in some embodiments 600° F., 700° F., 800° F., 900° F. and as much as 1,000° F. The operation of a system including the burner apparatus 106 is as described before. Pump 133 will pull air through burner inlet conduit 136 from air supply duct 104. Air will pass into interior 140 of silo 138 into the dirty air plenum 150. Air may be heated by a firebox or other heating mechanism in dirty air plenum 150. Air will be pulled through each of individual burner cylinders 158 and will be heated as it passes therethrough. The heated air stream will pass through outer housing 170 and into clean air plenum 152. A heated air stream will be communicated into a connecting conduit 177 and into a cooling apparatus 110 and UV chamber 114. Air will then be delivered into building 100 as previously described. The air will be heated to a temperature sufficient to eliminate, or at least significantly reduce pathogens from the contaminated air treated by the system 102. As explained below, tests have shown exposing air laden with MS-2 to high temperatures will eliminate, or at least dramatically reduce the MS-2 in the treated air. MS-2 is a bacteriophage that is accepted as a surrogate for SARS-CoV-2 and other pathogens and is more difficult to eliminate than SARS-CoV-2.

The temperature of each of burners 132 and 134 may be monitored and if the temperature to which the air is exposed in the burner 132, or if the temperature of the air leaving the burner falls below a specified temperature the valve 137 may be actuated so that the air from conduit 104 is redirected. For example, the air can be redirected from burner 132 to burner 134. The burner 132 will be heated to reach temperatures above the specified temperature as the contaminated air is directed to and heated by burner 134. The temperature at which the valve 137 will be actuated can be specified by the operator. For example, if the minimum desired temperature of the air leaving burner 132 is 500° F. and the temperature falls below 500° F., the valve 137 can be actuated to switch to burner 134. Rather than air temperature, the monitored temperature can be the internal temperature of the burner 132. This process can be continuous such that the temperatures to which the air is exposed and the air temperature leaving an individual burner can be monitored and the valve 137 actuated to switch back and forth between burners when the monitored temperature reaches a minimum specified temperature. Two burners are disclosed herein but it is understood that more than two may be utilized.

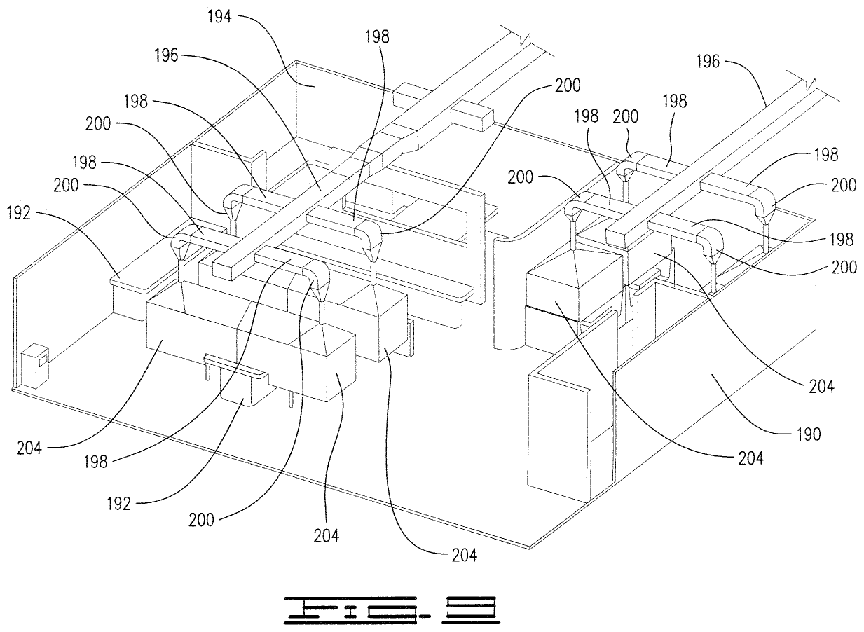

System 102, like the other systems disclosed herein, operate independent of existing HVAC systems, and are not connected thereto in any way. Although in the embodiment described in FIG. 5 the air treatment system 102 is used in an industrial setting where a plurality of work stations are included, the system may be used in other environments with human occupiable locations such as those shown for example in FIGS. 8 and 9. FIGS. 8 and 9 show an embodiment of a facility 190 that may be for example a restaurant or portion of an office building.

Facility 190 has a plurality of human occupiable locations 192. Facility 190 is shown without a wall and a roof so that the indoor space 194 is visible. Locations 192 may be work stations, eating locations, or other locations occupiable by a human. At least one air duct 196, and in the embodiment described a plurality of air ducts 196 are positioned in indoor air space 194 and have air duct branches 198 extending outwardly and downwardly therefrom. The air ducts 196 may be communicated with and comprise part of an air treatment system as described herein, like for example air treatment system 102. Contaminated air from indoor space 194 drawn into and treated by the air treatment system 102 will be communicated back into the indoor air space 194.

In FIG. 8 air duct branches 198 are hanging above human occupiable locations 192 with no additional conduit or updraft hoods. Air may be withdrawn from the indoor space 194 through air duct branches 198, into indoor air ducts 196 and into treatment system 102 through air supply duct 104 that will be connected to indoor air ducts 196. Air duct branches 198 have inlets 200 that communicate air drawn therein into indoor air ducts 196. Air inlets 200 may in turn be connected to updraft hoods 204 as shown in FIG. 9. Updraft hoods 204 are connected to inlets 200 with air conduits 206. Updraft hoods 204 are shown to completely enclose for example seating areas, work spaces, desk areas and other human occupiable spaces. Air expelled by humans in locations 192 will be pulled into updraft hoods 204, communicated into air supply duct 104 and treated by system 102 so that pathogens are removed therefrom, and treated air is exhausted back into indoor space 194. In the example of FIG. 9, all of the air expelled by persons in the updraft hoods will be pulled into updraft hoods 204 and treated by system 102.

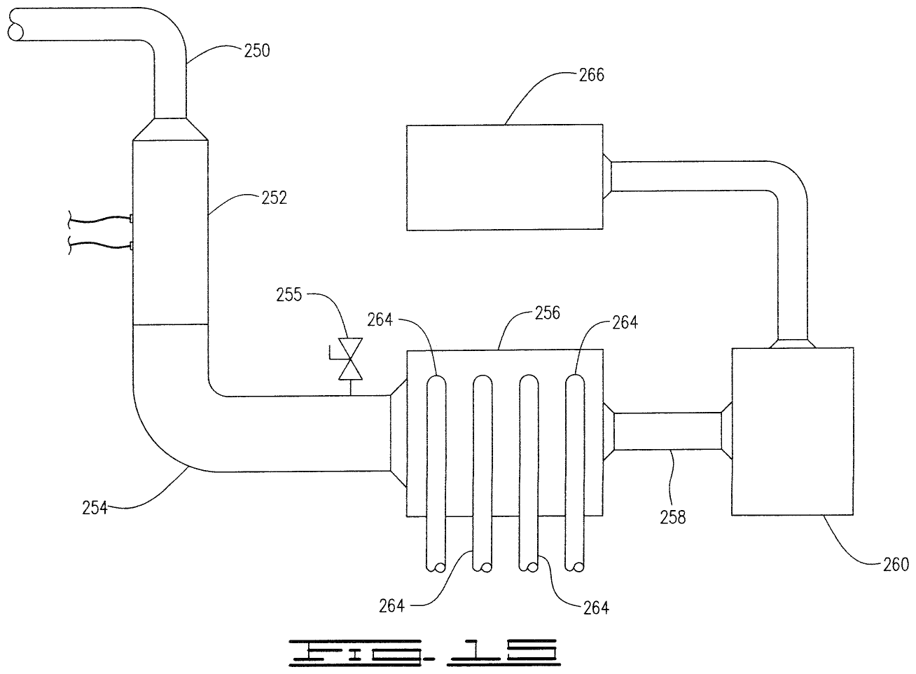

FIG. 14 shows another example of an environment in which the system might be utilized. Environment 300 may be a waiting room, or other contained area with indoor space 302. An updraft hood 304 is connected to an indoor air duct 306 that may be connected to and communicated with a system for treating air as described herein. Only one hood 304 is shown, but it is understood that a plurality of hoods 304 may be used.

Updraft hoods 304 are placed proximate a human occupiable space for example a chair in which a person may be seated. Air from indoor space 302 will be drawn into indoor air duct 306 and will be treated with a system as described herein. Air will be returned through a return air duct 308 connected to and communicated with the air treatment system. The air exhausted from return air duct 308 will contain fewer pathogens than the air drawn into hood 304. Air expelled by a person proximate the hood 304, or at least a portion thereof, will be pulled into hood 304, so that pathogens contained therein are prevented from entering the indoor space 302.

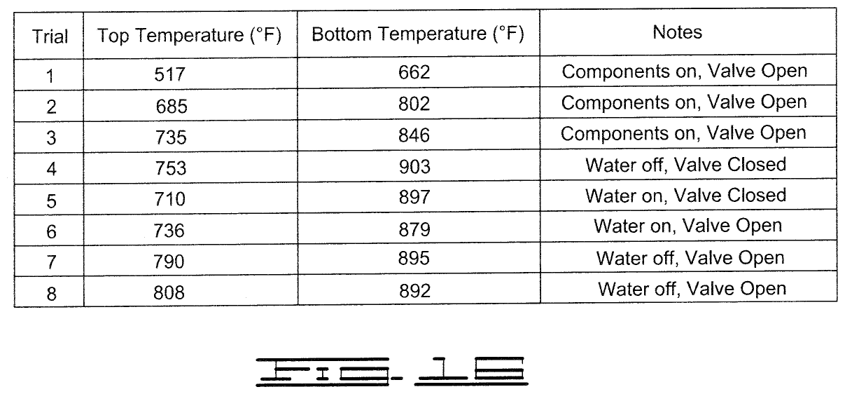

FIG. 15 shows a test setup establishing the efficacy of an air treatment system and method as described and claimed herein. The test arrangement included an inlet hose 250, a burner section 252 and a connecting conduit 254 communicating air passing through burner 252 to a cooling unit 256. A fresh air damper 255 was connected to connecting conduit 254. Cooling unit 256 may comprise a scrubbing unit as well. Cooling unit 256 has a plurality of inlets 264 through which water is sprayed so that air passing therethrough is cooled by the water. The water was in some cases continuously pumped through the cooling unit 256 as tests were run. A connecting conduit 258 delivered the air into a UV chamber 260 having a plurality of UVC lamps with ozone to expose the air passing therethrough to UV radiation. The air was then passed into a sample container 266 where the air was cultured to determine the existence of any viruses or other pathogens.

Test microorganisms were cultured, purified and concentrated prior to commencement. An MS-2 bacteriophage suspension was diluted to a starting concentration of 10⁶ plaque forming units (PFU) per milliliter. Aerosols including the MS-2 were generated for five minutes while the test device was running and air was pumped through the test system. An atomizer was connected directly to the input 250 aerosolizing approximately 1.67 mL of the viral suspension. The output of the test device was piped into an ASTM chamber located in a chemical fume hood. Samples were collected using single stage Anderson viable impactor placed into the ASTM chamber. The device was operated for five minute runs. Samples were collected with each run along with temperature readings. The number of viable microorganisms were determined quantitatively using plaque counting techniques and converted to PFU/M².

The burner section 252 of the test arrangement was a heating chamber of approximately four inches in diameter by twelve inches in length. The fill media in the heating chamber included 0.177 in. diameter metal beads heated by an electric heating coil. The inlet hose 250 was approximately ½ inch in diameter and air was pulled through the burner and through the system at approximately two cubic feet per minute. With a blower speed set at 2 cubic feet per minute or, 3456 cubic inches per minute within a volume of 0.087 cubic ft or, 150.72 cubic in and utilizing a magnehelic gauged vacuum reading of 0.75 inches the measured residence time of introduced continuous air plume was 2.61 seconds. In other words, the travel time for the air from the top of the burner to the bottom of the burner was 2.61 seconds. The UV chamber 260 included two T5 UVC tube light fixtures with ozone.

The results of eight trials are shown in FIG. 16. Tests were run with different components on and off to determine the impact. For trials 1, 2 and 3, the fresh air damper 255 was open and water was circulated through cooling unit 256. The top temperature is that temperature measured at the top of the burner 252 and the bottom is that temperature measured at the bottom of burner 252, For trial 4 no water was circulated and the damper 255 was closed. On trial 5 water was circulated in cooling unit 256 and the damper was closed. On trial 6 water was circulated through cooling unit 256 and the damper 255 was open, and for trials 7 and 8 the damper 255 was open and no water was circulated. Each trial indicated greater than a 99.1% reduction of MS-2 bacteriophage relative to the recovery on control samples. In effect, zero viable virus remained after treatment. Because it is known that the MS-2 bacteriophage is a surrogate for SARS-CoV-2 and is more difficult to inactivate, it may be concluded that the systems and methods described will effectively kill the SARS-CoV-2 and other pathogens.

In addition, there are studies indicating that elevated temperatures may be effective to kill. SARS-CoV-2. SARS-CoV-2 is one of a number of coronaviruses, one of which is SARS-CoV which is closely related to SARS-CoV-2. Different studies have indicated that most coronaviruses would be killed after exposure to 149° F. for longer than three minutes. For temperatures lower than 149° F. indications were that longer exposure times were needed. At least one study estimated that SARS-CoV-2 would be killed after an average of 2.5 minutes at 158° F. Although the studies appear to have considered the impact of elevated temperature on surfaces, the correlation between the temperature and exposure time indicates that the higher the temperature, the less exposure time is required to eliminate SARS-CoV-2. Thus, it is believed that exposing contaminated air to significantly elevated temperatures, for example temperatures of 500° F. and higher as noted herein, should decrease the exposure time needed to eliminate, or at least significantly reduce pathogens in the contaminated air.

Thus, it is seen that the apparatus and methods of the present invention readily achieve the ends and advantages mentioned as well as those inherent therein. While certain preferred embodiments of the invention have been illustrated and described for purposes of the present disclosure, numerous changes in the arrangement and construction of parts and steps may be made by those skilled in the art, which changes are encompassed within the scope and spirit of the present invention.

What is claimed is:

1. An air treatment system comprising:
an air duct separate from any existing HVAC system having at least one air inlet communicated with an indoor space;
a burner communicated with the air duct through which air from the air duct passes, the burner configured to heat the air received in the air duct to a temperature sufficient to reduce the pathogen load of the air passing therethrough;
a cooling apparatus communicated with the burner through which a heated air stream exiting the burner will pass to create a cool air stream having a temperature of not more than 10° F. higher than the ambient temperature of the indoor space;
a blower communicated with the air duct and configured to create a vacuum at the at least one air inlet to pull air from the indoor space into the at least one air duct; and
a return air duct separate from any existing HVAC system communicated with the cooling unit and having an air outlet communicated with the indoor space to deliver the cooled air stream from the cooling unit into the indoor space.

2. The air treatment system of claim 1, further comprising a UV chamber communicated with the cooling apparatus through which air passes prior to entering the indoor space, the UV chamber having a plurality of UV lamps positioned therein directly exposing the cooled air stream to UV radiation.

3. The air treatment system of claim 2, further comprising at least one updraft hood positioned proximate a human occupiable space such that air expelled by a human in the occupiable space will be pulled into the air duct through the updraft hood.

4. The air treatment system of claim 3, the updraft hood positioned to at least partially enclose a human in the occupiable space.

5. The air treatment system of claim 3, the at least one updraft hood comprising a plurality of updraft hoods.

6. The air treatment system of claim 2, the burner comprising:
an outer housing;
a fill media disposed in the outer housing, the air from the air duct communicated through the fill media; and
an induction coil positioned proximate the fill media.

7. The air treatment system of claim 2, the burner unit comprising:
a silo defining a dirty air plenum and a clean air plenum, the air duct communicated with the dirty air plenum;
a tube sheet disposed in the silo separating the clean air plenum from the dirty air plenum; and
a plurality of heating units suspended from the tube sheet, the heating units comprising:
an outer housing having an open top for receiving air from the dirty air plenum;
a fill media in the interior of the outer housing; and
a heating element proximate the filter to heat the fill media.

8. The air treatment system of claim 6, the outer housing comprising a porous ceramic body, the heating units further comprising at least one metal rod therein, the heating element comprising an induction coil.

9. The air treatment system of claim 1, the burner unit configured to generate an internal temperature of at least 662° F.

10. A method of treating air from an indoor space comprising:
positioning a plurality of updraft hoods in the indoor space;
drawing air from the indoor space into the updraft hoods;
heating the air drawn into the updraft hoods in a burner having an internal temperature of at least 662° F. to create a heated air stream;
cooling the heated air stream to a temperature of not greater than 10° F. above the temperature of the indoor air to generate a cool air stream; and
exhausting the cool air stream back into the indoor space.

11. The method of claim 10, further comprising exposing the cool air stream to UV radiation prior to the exhausting step.

12. The method of claim 10, the heating step comprising exposing air drawn into the updraft hoods in the burners for no more than three seconds.

13. The method of claim 10, the positioning step comprising positioning the updraft hoods proximate a plurality of human occupiable spaces.

14. The method of claim 13, further comprising at least partially enclosing humans in the occupiable locations with the updraft hoods.

15. The method of claim 10, the burner configured so that the maximum residence time of the indoor air in the burner is less than about three seconds.

16. The method of claim 10, further comprising heating the indoor air with the burner to a temperature sufficient to kill the SARS-CoV-2 virus.

17. A method of treating air from an indoor space comprising:
positioning a plurality of updraft hoods in the indoor space;
at least partially enclosing humans in at least a portion of the plurality of updraft hoods;
connecting the updraft hoods to an air duct;
pulling air expelled by the humans positioned in the updraft hoods into the air duct;
heating the air expelled by the humans in the updraft hood to a temperature sufficient to reduce the pathogen load in the expelled air;
cooling the air after the heating step to a temperature of not greater than 10° F. above the temperature of the indoor air to generate a cool air stream; and
exhausting the cool air stream back into the indoor space.

18. The method of claim 17, the heating step comprising exposing the air from the air duct to a temperature of at least 662° F.

19. The method of claim 17, the heating step comprising passing the air from the air duct through a burner.

20. The method of claim 19, the burner configured so that the residence time of the air in the burner is no more than three seconds.

21. The method of claim 17 further comprising exposing the cooled air stream to UV radiation prior to exhausting the cool air stream back into the indoor space.

22. A system for treating air from an indoor space comprising:
an air duct;
a plurality of updraft hoods communicated with the air duct and positioned proximate human occupiable spaces in the indoor space;
a burner having an internal temperature of at least 500° F. communicated with the air duct;
a cooling apparatus communicated with the burner to cool the air from the heater to a temperature of not higher than 10° F. above a temperature of the air in the indoor space;
a UV chamber communicated with the air exiting the cooling apparatus;
a blower communicated with the UV chamber configured to pull air from the updraft hoods through the burner, cooling apparatus and UV chamber at a rate such that the residence time for air passing through the burner is no greater than three seconds; and
an exhaust duct communicated with the blower, the exhaust duct having an outlet for exhausting air communicated from the blower into the indoor space.

23. The system of claim 22, the heater comprising:
an outer housing;
a heatable media filling at least a portion of the outer housing for the passage of air therethrough; and
a heater coil proximate the media.

24. The system of claim 22, further comprising:
a plurality of burners; and
a switch valve connected to the burners and configured to periodically redirect the indoor air from one of the plurality of heaters to another of the plurality of heaters.

25. The system of claim 22, the updraft hoods comprising an outer wall having a bottom opening, the updraft hood being movable from a raised position to a lowered position to enclose at least a portion of a person in an occupiable space.

26. The system of claim 22, the updraft hoods comprising a cubicle with an opening in the outer wall to allow a human to enter the cubicle.
